(19) United States
(12) Patent Application Publication
(10) Pub. No.: US 2023/0414309 A1
(43) Pub. Date: Dec. 28, 2023

(54) SURGICAL TOOL SUPPORT SYSTEM

(71) Applicant: THE UAB RESEARCH FOUNDATION, Birmingham, AL (US)

(72) Inventors: Kyle Wood, Vestavia Hills, AL (US); Andres Morales, Birmingham, AL (US); Sean Simmons, Birmingham, AL (US)

(21) Appl. No.: 18/265,080

(22) PCT Filed: Dec. 7, 2021
(86) PCT No.: PCT/US2021/072777

§ 371 Date: Jun. 2, 2023

Related U.S. Application Data
(60) Provisional application No. 63/122,068, filed on Dec. 7, 2020.

(51) Int. Cl. A61B 50/26; A61G 13/10

(52) U.S. Cl. CPC A61B 50/26; A61G 13/101

(57) ABSTRACT

A surgical tool support system is provided. The system includes a support portion, a swivel arm, and a tool holder for holding a surgical tool coupled to a distal end of the swivel arm. The tool holder can have a locking joint controlled by an actuator such that the tool holder can be locked into a position along multiple axes of rotation. The system can offset the force required by a user to move the surgical tool, thereby reducing fatigue.

### CROSS-REFERENCE TO RELATED APPLICATIONS

This application claims the benefit of and priority to U.S. Provisional Application Ser. No. 63/122,068, having the title “SURGICAL TOOL SUPPORT SYSTEM”, filed on Dec. 7, 2020, the disclosure of which is incorporated herein by reference in its entirety.

### BACKGROUND

Damage to surgical scopes such as ureteroscopes is costly, as are the disposable scopes used as an alternative. Additionally, handling of scopes is difficult when performing other surgical tasks and can contribute to surgeon fatigue and increase risk of patient injury from mishandling. The scope may also be put down when not in use during a procedure and placed in locations that compromise sterility. Finally, the current surgical field during endoscopy is not optimized for the procedure nor the surgeon.

### SUMMARY

Embodiments of the present disclosure provide for surgical support devices and systems, surgical tool holders and the like.

An embodiment of the present disclosure includes a surgical tool support device including a vertical support pole and a static arm coupled perpendicularly to the support pole at a first end. A proximal end of a swivel arm is coupled to a second end of the static arm. A tool holder is coupled to a distal end of the swivel arm. The tool holder can include a locking joint controlled by an actuator such that the tool holder can be locked into a position along multiple axes of rotation.

An embodiment of the present disclosure also includes a surgical tool holder including a clamp in communication with an actuator via a locking mechanism. The clamp is freely rotatable around an axis of the tool holder when the actuator is not actuated. When the actuator is actuated, the clamp is locked into position by a locking mechanism.

An embodiment of the present disclosure also includes a system for surgical tools. The system can include a surgical tool support device as above, and at least one attachment. The surgical tool support device provides a counter force equal to the force required by a user to position the surgical tool.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a diagram illustrating a surgical tool support system including a ball and socket joint brake in accordance with embodiments of the present disclosure.

FIGS. 2A-2C are diagrams illustrating a magnetic ball and socket joint brake in accordance with embodiments of the present disclosure.

FIGS. 3A and 3B are diagrams illustrating the operation of the magnetic ball and socket joint brake in accordance with embodiments of the present disclosure.

FIGS. 4A-4C are diagrams illustrating a mechanical ball and socket joint brake in accordance with embodiments of the present disclosure.

FIGS. 5A-5D are diagrams illustrating the operation of a mechanical ball and socket joint brake in accordance with embodiments of the present disclosure.

FIG. 6 is a diagram of a scope holder in accordance with embodiments of the present disclosure.

FIGS. 7A-7D are diagrams illustrating a surgical tool support system that provides a user with weightless articulation of a surgical tool in accordance with embodiments of the present disclosure.

FIGS. 8A-8D are diagrams illustrating a surgical tool support system according to embodiments of the present disclosure shown in reference to a patient.

FIGS. 9A-9B provide a front view and front cross-section view of a rotatable surgical tool holder in accordance with embodiments of the present disclosure.

FIGS. 10A-10D provide a side view, side cutaway view, side cross-section view, and perspective cutaway view, respectively, of a rotatable surgical tool holder in accordance with embodiments of the present disclosure.

FIGS. 11A-11D provide detail views of a surgical tool holder in accordance with embodiments of the present disclosure.

FIGS. 12A-12C are diagrams illustrating the counterbalancing mechanism of the surgical tool support system according to embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of physics, engineering, manufacturing, which are within the skill of the art.

It must be noted that, as used in the specification and the appended claims, the singular forms “a,” “an,” and “the” include plural referents unless the context clearly dictates otherwise.

In accordance with the purpose(s) of the present disclosure, as embodied and broadly described herein, embodiments of the present disclosure, in some aspects, relate to surgical support tools including locking ball and socket joints, and locking ball and socket joints.

The present disclosure includes a surgical tool support system. The surgical tool support can include a vertical support pole and a static arm having a rigid sleeve. The rigid sleeve slidably affixes a first end of the static arm perpendicular to the vertical support pole. The system also includes a motor brake and a swivel arm, wherein a proximal end of the swivel arm is coupled to a second end of the static arm. A first locking ball and socket joint connects a distal end of the swivel arm to a proximal end of a first movable arm. A second locking ball and socket joint connects a distal end of the first movable arm to a proximal end of a second movable arm. A tool holder can be connected to a distal end of the second movable arm. The motor brake activates or deactivates the first and second locking ball and socket joints.

### General Discussion

The present disclosure provides for devices and systems for holding and supporting surgical tools, such as a scope. The devices and systems allow a user to position and rotate the scope with little to no exertion of force, as the force is offset by the device. The tool can be locked into position via an actuator. Advantageously, the devices and systems described herein can provide a significant reduction in surgeon fatigue, as well increasing both efficiency and sterility of a procedure. The tool can be maneuvered with an ease and range of motion similar to a joystick.

Embodiments of the present disclosure include a locking ball and socket joint, wherein the locking ball and socket joint is an electromagnetic locking ball and socket joint. In some embodiments, the joint includes a metal ball comprising a threaded screw and a magnetic socket to receive the metal ball and an electromagnet, where the threaded screw connects the joint to the movable arm. As can be envisioned by one of ordinary skill in the art, the ball can be non-metallic, e.g. plastic, ceramic, or other suitable material. In some embodiments, the ball can be connected to the movable arm by other fastening means, including but not limited to a pressure-fitted rod, welding, gluing, twist lock, etc. The joint also includes a casing.

Embodiments of the present disclosure also include a locking ball and socket joint as above, wherein the locking ball and socket joint is a mechanical locking ball and socket joint. In some embodiments, the mechanical locking ball and socket joint includes a metal ball having a threaded screw and a socket to receive the metal ball. The joint also includes a servo motor attached to a screw and a casing comprising a hole to receive the screw. As can be envisioned by one of ordinary skill in the art, the ball can be non-metallic, e.g. plastic, ceramic, or other suitable material. In some embodiments, the ball can be connected to the movable arm by other fastening means, including but not limited to a pressure-fitted rod, welding, gluing, twist lock, etc.

Embodiments of the present disclosure includes a surgical tool support device including a vertical support pole and a static arm coupled perpendicularly to the support pole at a first end. A proximal end of a swivel arm is coupled to a second end of the static arm. A tool holder is coupled to a distal end of the swivel arm. The tool holder can include a locking joint controlled by an actuator such that the tool holder can be locked into a position along multiple axes of rotation.

Embodiments of the present disclosure include a surgical tool holder including a clamp in communication with an actuator via a locking mechanism. The clamp is freely rotatable around an axis of the tool holder when the actuator is not actuated When the actuator is actuated, the clamp is locked into position by a locking mechanism.

Embodiments of the present disclosure also include a system for surgical tools. The system can include a surgical tool support device as above, and at least one attachment. The surgical tool support device provides a counter force equal to the force required by a user to position the surgical tool.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

A wide variety of endoscopic procedures are performed every day around the world. Generally, endoscopic procedures involve a viewing scope, referred to generally as an endoscope, that is inserted through an opening (e.g. an orifice of the subject or a wound specifically generated for insertion of the endoscope into the subject) and into the subject to allow visualization of internal portions of the subject. Endoscopes generally include an illumination source and can a camera to allow visualization of the internal portion of the subject. There are specific types of endoscopes configured for specific procedures. For example, a laparoscope is a type of endoscope that is designed for laparoscopic (e.g. abdominal) procedures and arthroscopes are those designed for arthroscopic procedures (e.g. joint) procedures. Another example is an ureteroscope, used by a urologist to visualize the urinary tract. Although they can vary, they all can have the same general features and purpose of providing a view inside the body.

During an endoscopic procedure, the medical practitioner (e.g. a surgeon) and assistants are involved in performing the endoscopic procedures to manage the endoscope, ports, and other devices used during the procedure. Some scopes are designed to be rigid while others are flexible; flexible scopes often designed with more complexity to allow for movement of the device. Some scopes have working channels allowing for passage of instruments such as graspers, lasers, and wires.

In some instances, an assistant is needed just to manage the endoscope during the procedure or assist in passage of devices through the working channel, which can increase the number of personnel required for a procedure. Further these endoscopes are costly instruments and can require significant repair after only a few procedures. For some procedures, such as a ureteroscopy, it is estimated that new digital endoscopes cost about $20,000 and more than $6,000 per repair, which are typically required about every 12 cases.

Ureteroscopes are flexible devices with operator-controlled movements. In addition, newer scopes have state of the art optics. These features in concert with their compact engineering can result in significant repair costs. The literature estimates that repair costs for this particular type of endoscope per procedure is about $700-$1,000. Moreover, endoscopes can be placed in locations during the procedure that can compromise sterility, which can increase the risk of patient infection. Finally, endoscopic devices can be heavy, which can result in physician fatigue during longer procedures.

In view of these problems with current endoscopes and their use, described herein is a surgical tool support system that can attach to a medical table, bed, or chair (e.g. surgical, hospital, procedure, exam or otherwise) or as a stand-alone device and can be configured to hold an endoscope or other tool (e.g. precision hand tools) while allowing for its operation and use during a procedure. In some embodiments, the support system can be freestanding on a movable base, similar to an IV stand.

Advantageously, the surgical tool support system when used with an endoscope can reduce the number of assistants required for an endoscopic procedure, optimize the surgical field, can reduce the risk of infection, and/or protect the scope during a procedure, which can reduce the cost of the procedure to the subject because it can reduce the number of repairs needed during the lifetime of the endoscope. Other devices, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Despite antibiotic use, 2% of ureteroscopy cases are complicated by infection resulting in prolonged hospital stay and thus increased cost. Current scopes are placed in locations during case that compromise sterility. The surgical tool support system described herein can allow for a more sterile procedure, reduction in unnecessary infections and complications, and increased patient safety.

The surgical tool support system can serve as “an extra hand”. During surgical procedures such as stone removal, multiple actions are required including using lasers, baskets, and wires that must be passed through working channels. Handling of scopes can become difficult during times of working channel utilization. Case duration can span many hours and the scopes become heavy, resulting in physician fatigue. The surgical tool support system of the present disclosure can decrease reliance on an assistant and allow the surgeon to control the passage of baskets, wires, and lasers with more precision. In addition, the surgical tool support system is ergonomic and has free mobility (within all directions) within the operative field but can be locked in place with a simple push of a button to secure the scope.

### Example 1

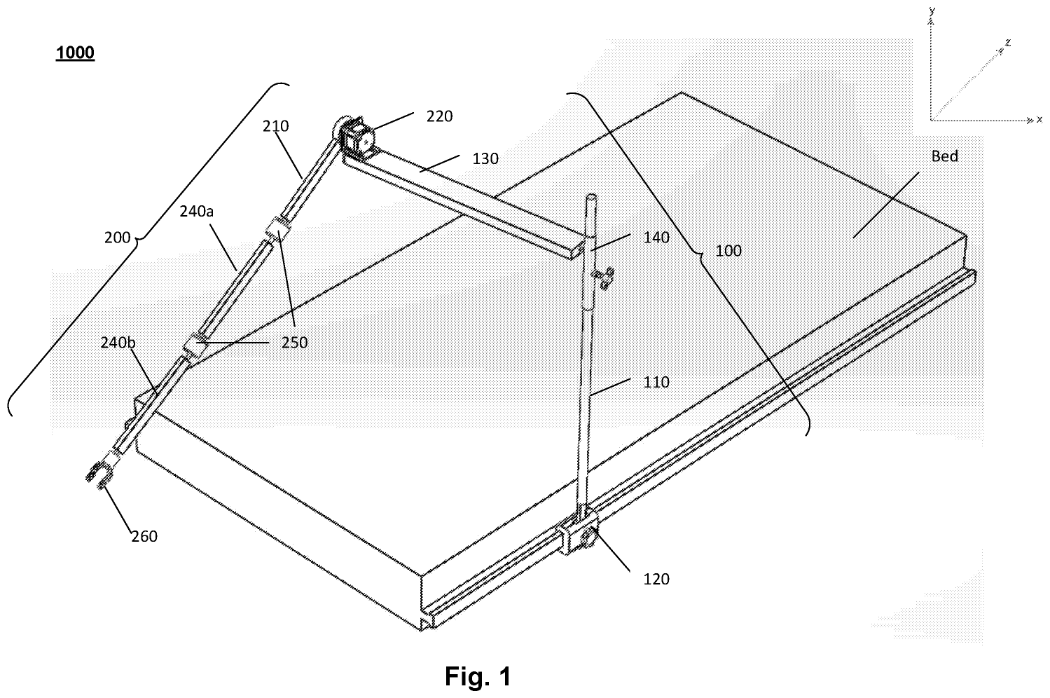

The present disclosure includes several embodiments of surgical tool support systems (also referred to as endoscope support systems). Turning to the drawings, FIG. 1 is a diagram illustrating a tool support system 1000 including locking ball and socket joints in accordance with embodiments of the present disclosure. The surgical tool support system includes a swivel arm 210 and motor brake 220. In general, the system includes a support portion 100 and a movable portion 200 that can be controlled by the operator (e.g. a surgeon). The support portion 100 includes a vertical support pole 110 connected to a static arm 130. The static arm 130 includes features for attachment of a motor brake 220. The movable portion includes a swivel arm 210 connected to a movable arm 240a or series of movable arms (collectively referred to as movable arms 240) via locking ball and socket joints 250. The distal-most movable arm 240b can end in a tool holder 260. Further description of the locking ball and socket joints 250 is provided in the discussion of the figures.

When the system is not energized, the swivel arm 210 and movable arms 240 are free to move in a variety of positions. When the movable portion 200 is in a position desired by the operator, the system can be energized, and the movable portion 200 locked into position by the motor brake 220 and locking ball and socket joints 250. Advantageously, this leaves the tool locked in place and the operator free to perform other tasks while maintaining a sanitary environment for the tool.

In some embodiments, the support pole 110 can be attached to a bed having rails by a clamp 120. The clamp 120 can be adjusted to fit a number of bed models, and the support pole 110 can be positioned at various positions along the bed. In other embodiments, the support pole 110 can be fitted on a movable or wheeled base, similar to an IV stand, such that the entire support system can be freestanding. The movable base can include locking features, such as locking casters to prevent movement of the base while in use. The support pole 110 can have a different geometry on each end to adapt to different types of clamps (e.g. socket or blade clamp). Advantageously, the support system can be used with virtually any existing setup in an operating theater or clinic.

A static arm 130 can be connected to the support pole 110 such that the height of the static arm 130 can be adjusted along the support pole 110 and the static arm 130 can be rotated about the y-axis, then fixed into place. The static arm 130 extends perpendicular to the upright support pole 110. In some embodiments, the static arm 130 can be rigidly connected to a sleeve 140 that slides over the support pole 110 for sterility and stability. The sleeve 140 can be rotated about the support pole 110 and then secured (e.g. with such as a thumbscrew) after initial positioning. In some embodiments, the static arm can be comprised of rails.

The static arm 130 includes fittings (e.g. a bracket) for attachment of the motor brake 220. The bracket can rotate around the y-axis of the static arm 130 for initial positioning.

The swivel arm 210 is connected to the motor brake 220. When the system is not energized, the swivel arm 210 is free to move. In contrast, when the system is energized, the motor brake 220 will hold the swivel arm 210 in place. The swivel arm 210 can rotate around the x-axis in a full 360 degrees. In alternative embodiments, the swivel arm 210 can be locked with another mechanism (e.g. brake caliper, friction pad, etc.).

The swivel arm 210 is connected to a movable arm 240 via a locking ball and socket joint 250 at the proximal end of the movable arm 240a. Additional movable arms 240 can be added distally via ball and socket joints to create a segmented movable arm 240 having multiple joints. Advantageously, the number of movable arms 240 and locking ball and socket joints 250 may change to accommodate surgical fields and/or surgeon's preference. In a particular embodiment, individual movable arms 240 may be about three feet in length. The movable arms 240 can be freely rotated along all three axes, providing the operator with precise control of positioning. The degrees of rotation available depends upon the number and length of movable arms 240 in the system.

The distal end of the final movable arm 240 can be connected to a tool holder 260. In some embodiments, the tool holder 260 is configured to be a scope holder. The tool holder 260 can be adjustable to fit different tools, or varying brands and sizes of scopes. In some embodiments, the tool holder 260 can be disposable. The tool holder 260 can be flexible and substantially U-shaped, such that it is under tension and expands to grasp the tool when the tool is inserted between the two arms. Other means of tension, such as spring loading can be envisioned by one of ordinary skill in the art. In some embodiments, the tool holder 260 can be tightened. For example, the tool holder can include two parallel holes configured to receive a screw and nut or other means of achieving an adjustably tightened hold. In some embodiments, the tool holder 260 can include a gel or compressible foam to protect the scope from damage.

The movable portion 200 of the surgical tool support system can be locked into a desired position by the operator by a button or a foot pedal. The button can be placed on or near the tool holder 260, and in some embodiments, can be overridden by the foot pedal.

The surgical tool support system can be powered by either a battery or via a wall outlet, with power cables running through the support pole 110, static arm 130, and movable arms 240 to power the logic circuit for the motor brake 220 and the locking ball and socket joints 250.

The locking ball and socket joint 250 functions as both a joint and a brake. The locking ball and socket joint 250 can be electromagnetically or mechanically operated. Advantageously, the locking ball and socket joints described herein can be used in a variety of other applications in which a large degree of freedom of movement coupled with the ability to lock into place is desired, including robotics or any device with movable arms. The locking ball and socket joints can be scaled up or down, depending on the size of the device in which they are used.

The surgical tool support system, the system can be easily cleaned and sterilized. The arms and other components can be protected by sleeves to reduce the risk of infection. Such sleeves can be added at the time of the procedure to ensure sterility. The arms and support pole can be hollow to protect the electrical components and cables, allowing for the external surfaces to be cleaned following sterile operating room protocols. The surgical tool support system can be modular, allowing for disassembly to facilitate thorough cleaning and/or autoclaving. Additionally, some components may be disposable, avoiding the need for post-procedure sterilization.

FIG. 1 provides one possible embodiment of the surgical tool support system. In this example, the support pole 110 is attached to a bed having rails by a clamp 120. The clamp 120 can slide across the bed rail along the x-axis to adjust the initial position of the support pole 110. The clamp 120 is secured by a thumbscrew. The static arm 130 is adjustably affixed to the support pole by means of a sleeve 140 that slides over the support pole 110 and can be rotated about and tightened onto the support pole 110. The height of the static arm 130 can also be adjusted along the support pole 110. The static arm 130, shown extending over the bed, includes a motor brake 220.

The swivel arm 210 is connected to the motor brake 220 at its proximal end. At the distal end of the swivel arm 210, a locking ball and socket joint 250 connects a first moveable arm 240. A second movable arm 240 is distally connected to the first movable arm 240a by a second locking ball and socket joint 250. Two movable arms 240 are shown in this particular embodiment, but a varying number of arms can be included, as can be envisioned by one of ordinary skill in the art. The movable arms 240 can have the same length or can have different lengths from one another. The distal end of the final movable arm 240b is connected to a tool holder 260. The tool holder 260 can be screwed into the movable arm 240 or otherwise removably connected. In some embodiments, the tool holder 260 can be rotated independently of the movable arm 240. In other embodiments, the rotation of the movable arm 240 determines the angle at which the tool holder 260 is rotated.

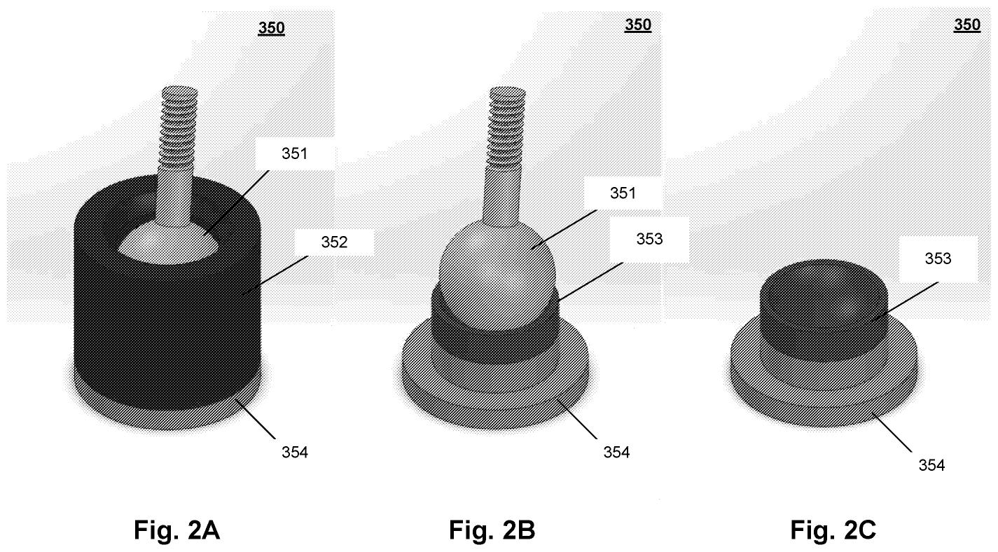

FIGS. 2A-2C provide an embodiment of locking ball and socket joint 250. The depicted embodiment is an electromagnetic ball and socket joint 350. The joint functions as both a joint and a brake for the moveable arms. When the electromagnet 354 is not energized, the magnetic socket 353 can rest against the electromagnet 354 and the ball 351 is free to move. When the electromagnet 354 is energized, the ball is locked into position, creating a brake.

The metal ball 351 rests in magnetic socket 353. The ball 351 includes a threaded screw to attach to the end of a movable arm 240. The magnetic socket sits atop electromagnet 354. The casing 352 encloses ball 351 and magnetic socket 353 to protect the joint and insulate the magnet. The shape of casing 352 also locks the ball 351 into position when the locking ball and socket joint 250 is in an energized state.

In some embodiments, the opposing poles of the magnetic socket 353 and the electromagnet 354 are facing one another. The magnetic socket 353 can be below the ball 351 as shown, locking when repelled. In other embodiments, the magnetic socket 353 can be above the ball 351, locking when the ball is attracted to the electromagnet.

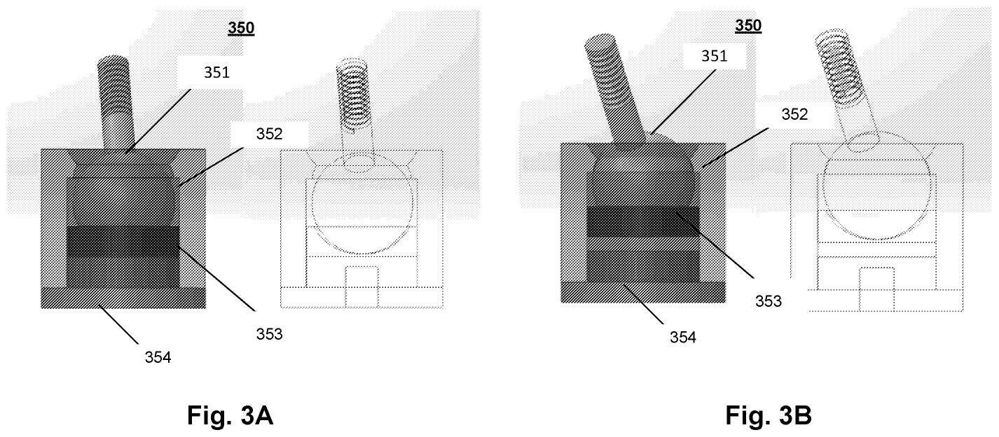

FIGS. 3A and 3B are diagrams illustrating the operation of the electromagnetic ball and socket joint brake 350. As described above, the overall surgical tool support system is powered by a battery or wall outlet connected to the motor brake 220. The motor brake 220 in turn activates or deactivates the electromagnetic ball and socket joint brake 350. When the electromagnet 354 is not activated, the magnetic socket 353 can rest against the electromagnet 354 and the ball 351 is free to move.

The polarities of the magnets are set such that when the electromagnet 354 is energized, the magnetic socket 353 is repelled. Therefore, the ball is pressed against the case and friction restricts the movement of the ball. When the electromagnetic ball and socket joint brake 350 is not energized, the ball 351 is free to move. When the electromagnetic ball and socket joint brake 350 is energized, the movement of ball 351 is restricted. The activation or deactivation of the electromagnetic ball and socket joint brake 350 can be controlled by the operator at the touch of the button or foot pedal.

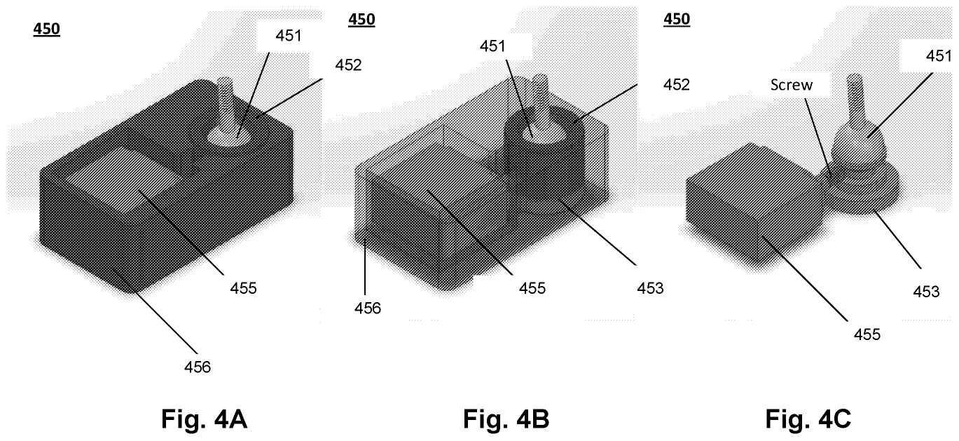

FIGS. 4A-4C illustrate another embodiment of locking ball and socket joint 250. The depicted embodiment is mechanical ball and socket joint 450. As above, the joint functions as both a joint and a brake for the moveable arms. The ball 451 rests in mechanical socket 453. The ball 451 includes a threaded screw to attach to the end of a movable arm 240. The casing 452 includes a threaded hole to accommodate a screw 454 attached to servo motor 455. Holder 456 houses the servo motor 455 and the casing 452.

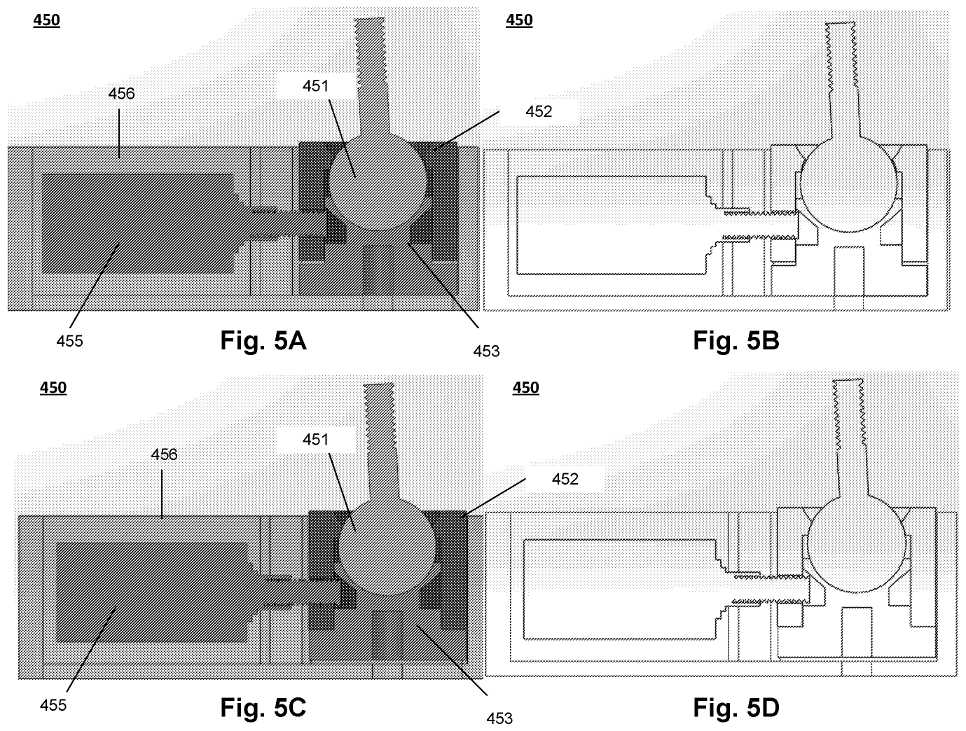

FIGS. 5A-5D are diagrams illustrating the operation of the mechanical ball and socket joint 450. The holder 456 maintains the position of the ball 451 and socket 453, and also prevents rotation of the servo motor 455. When the system is not activated (FIGS. 5A and 5B), the screw 454 attached to servo motor 455 is in a retracted position (note the small gap between casing 452 and socket 453). The socket 453 is in an inactivated position and the ball 451 is free to rotate. When the system is activated (FIGS. 5C and 5D), the servo motor 455 drives screw 454 into an extended position, pushing it through the hole in casing 452 towards the socket 453. The geometry of the socket 453 causes the socket to move upward as the screw 454 extends, thereby pressing ball 451 against casing 452. Friction restricts the movement of the ball 451.

### Example 2

Ureteroscopy is a procedure that uses a ureteroscope to look inside the ureters and kidneys, as well as to deliver tools via the scope probe to the internal sites requiring subsequent procedure(s). As discussed above, a ureteroscope is a hand-held, hand operated device with a camera on the distal end of the scope's probe with which the surgeon navigates to internal sites to locate and acquire insights into the areas of concern. This process of navigating the probe can require considerable lead times. Once the surgeon has located and accessed the issues and decides upon a course of remedy, the surgeon must load the scope with tools and attachments required to surgically address the condition. This requires the surgeon to set the scope down while they retrieve and load the tools/attachments.

After the surgeon has navigated the tip of the probe to the site of the health-issue by way of articulating the scope handle in space, the surgeon must set the scope down in order to retrieve the tools/attachments they determine are required for the ensuing surgical procedure(s). Then the tools/attachments are loaded into the scope. However, there are typically no sterile or available surfaces for secure placement of the scope for the required adjustments to the scope. This condition leads to the draping of the scope over the patient's leg, or over the surgical drape/bucket, where the scopes are often dropped or the flexible probe is inadvertently flexed beyond its limits resulting in costly damage to the probe or scope handle.

Additionally, the initial process of navigating the probe tips to the objective surgical sites is a tedious and fatiguing activity. Frequently, the site location must be subsequently reacquired after the tool loading activities due to the scope movement. These added steps contribute to user fatigue and extended surgical window requirements.

The devices and systems provided herein can alleviate or eliminate the problems related to damage and costs to the scope handle or probe, the need for reacquisition of the target surgical location, and dynamic muscular fatigue brought on by hours of unassisted hand-held use of the scope. While the example provided below refers mainly to ureteroscopes, other scopes or tools needed for other procedures can be accommodated.

Several embodiments of surgical tool support devices and systems (also referred to as endoscope support systems) are provided. The terms tool and scope can be used interchangeably, and it will be understood by one of ordinary skill in the art that other surgical tools can be substituted for a scope.

In general, the device is a surgical tool support device that can hold a variety of off-the-shelf tools such as a scope such that the user (e.g. a surgeon) can position the tool accurately within the procedure field and lock the tool into place to free the user's hands. The tool holder portion of the device allows for freedom of rotation of the tool for precision positioning with little resistance.

The device can also include a mechanical means that provides the user with a zero-or near-zero gravity weightless articulation of the scope to virtually eliminate user fatigue. In other words, the surgical tool support device reduces or removes the resistance and weight experienced by the user when maneuvering the tool. The mechanism allows for placement and support for the scope during the loading and fitting of the tool. This reduces or eliminates the need to place the scope in undesirable/unstable locations within the operational environment, thus substantially reducing the factors that lead to the damage of the scope/probe.

The device is also equipped with a locking means that allows the surgeon to initiate a positional lock as soon as they have acquired the desired position/location for the tool (e.g. the probe end of the scope). This feature can reduce or eliminate the need to reacquire the desired position after prepping the scope for the procedure.

In some embodiments, the surgical tool support device can include a tool holder having tool seat. The tool seat can be a spherical cradle that seats the probe and connects it to the device boom (also referred to as a static arm) by way of a ball and socket configuration. This ball-and-socket configuration allows the tool to attach to the device's boom with the axial freedom needed to place the tool, and in the case of a scope, to articulate the scope's probe end. The user can feed the scope's probe into the spherical scope cradle and the device can remain engaged while performing the procedure.

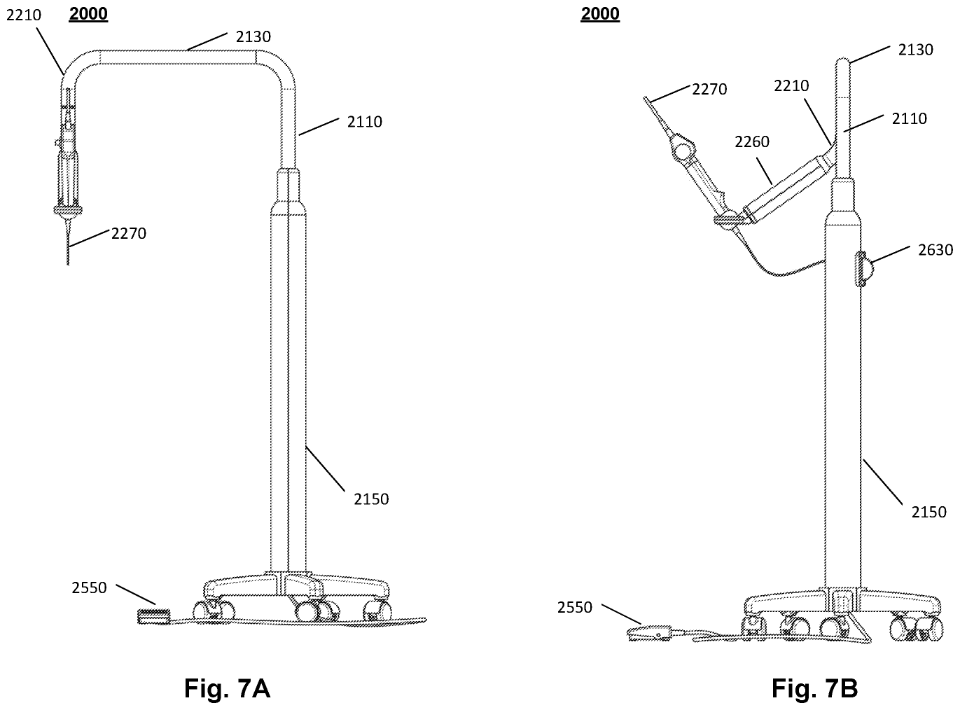

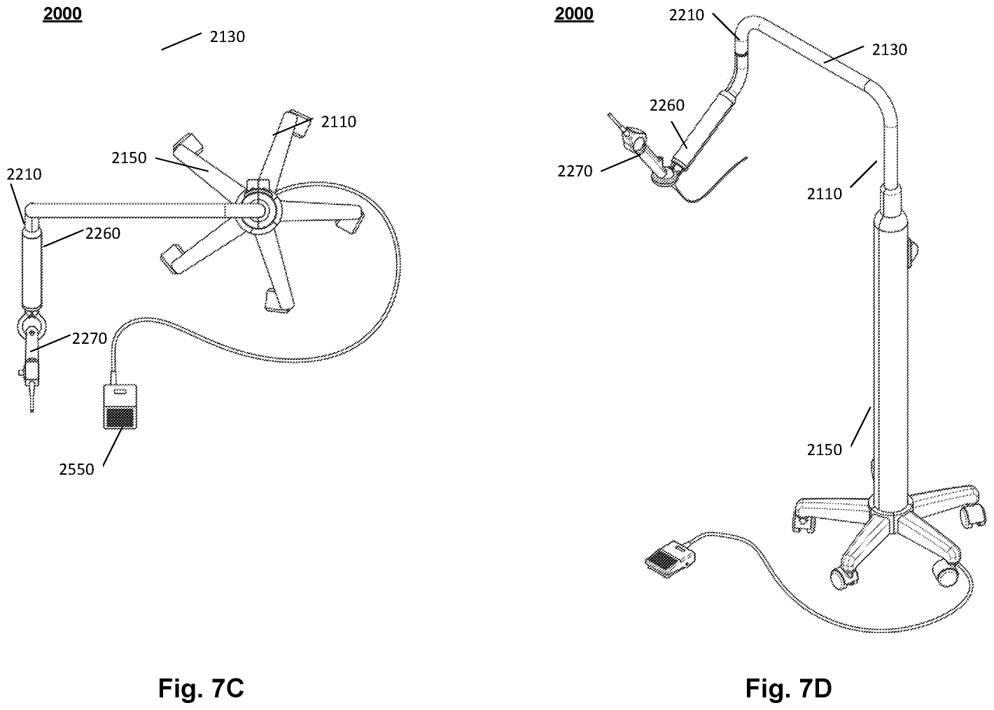

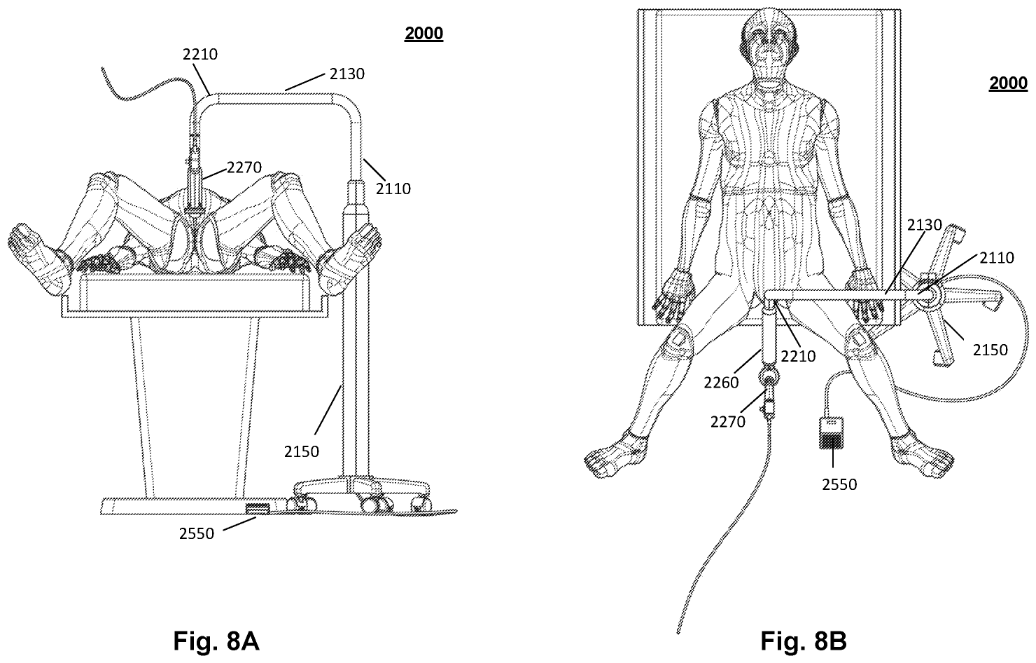

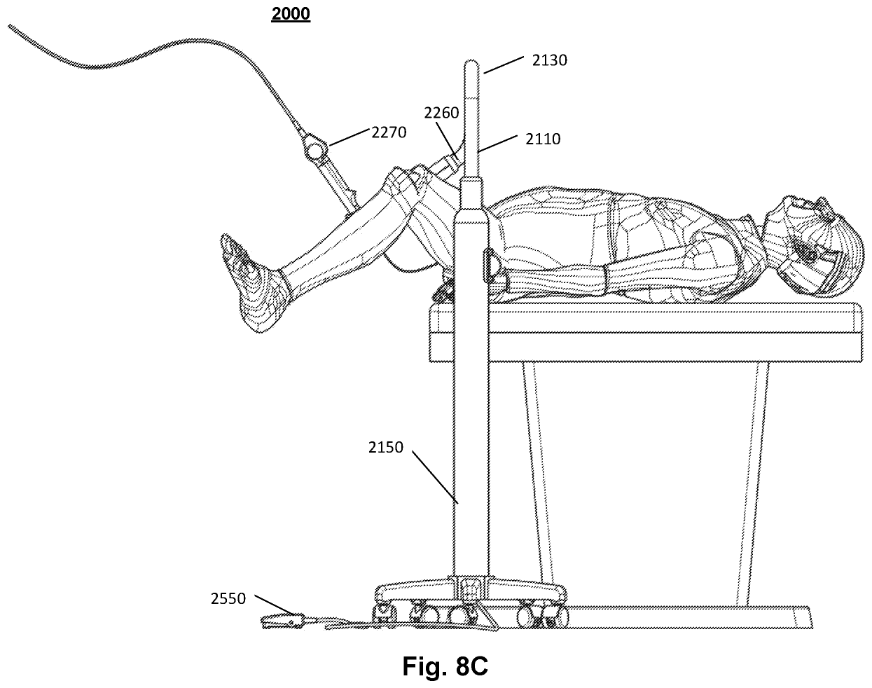

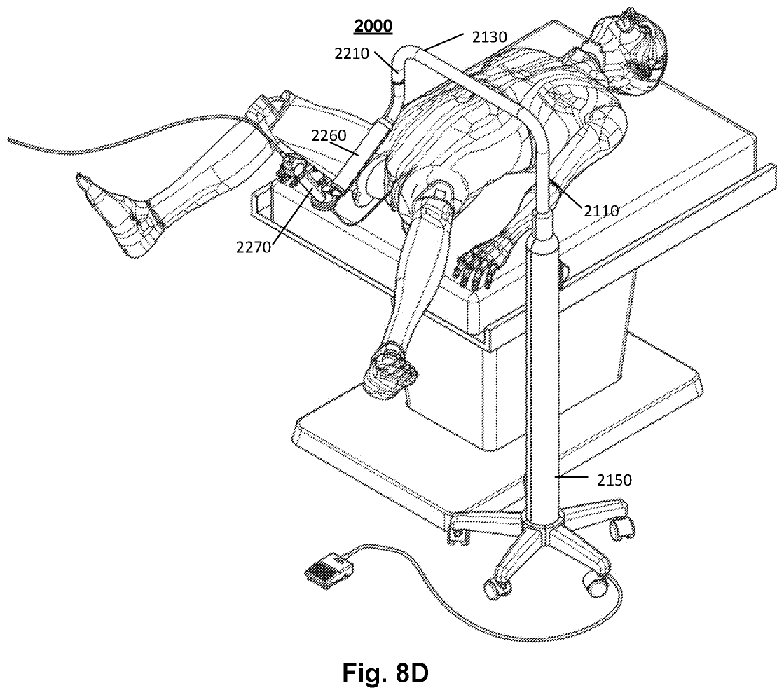

Embodiments of the device can be understood and appreciated by way of the drawings. Turning to the drawings, FIGS. 7A-7D are diagrams illustrating one possible embodiment of surgical tool support device 2000 that provides a user with weightless articulation of a surgical tool 2270. FIG. 7A is a front view of the device. FIG. 7B is a side view of the device. FIG. 7C shows the device from the top. FIG. 7D is a perspective view of the device. FIGS. 8A-8D illustrates the device in relation to a patient undergoing a ureteroscopy procedure from the front, top, side, and perspective views, respectively.

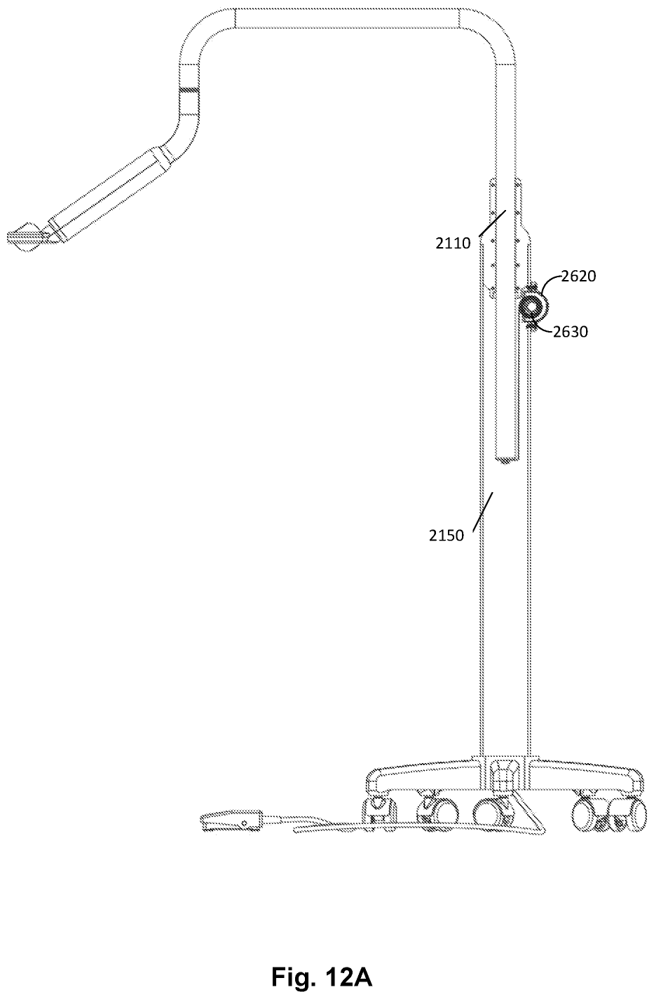

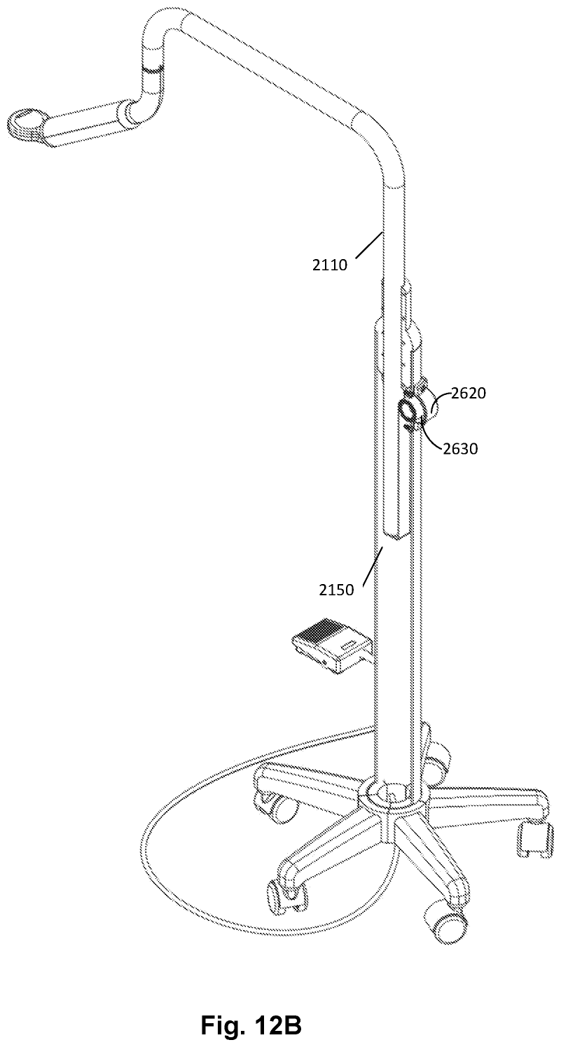

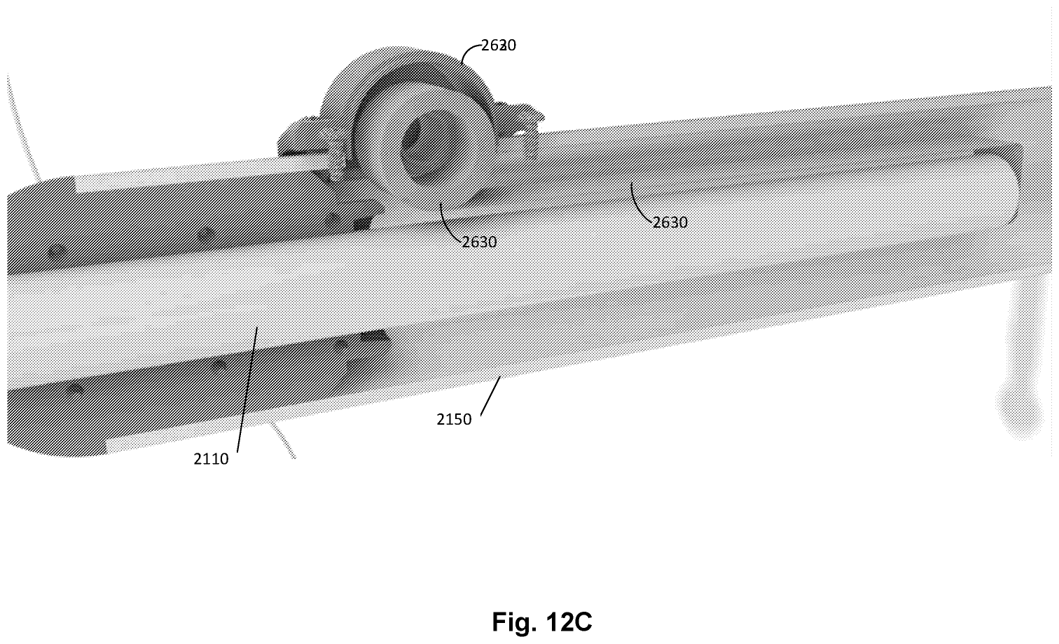

The device can have a vertical support pole 2110, which operates as an articulating boom. The support pole 2110 can raise and lower within a base 2150 along the x-axis and can rotate about the y-axis. The base 2150 can be a portable rolling base as shown, or can be a base configured to attach to the edge of a table or bed. In the case of the rolling base, the support pole can also be moved in the y- and z-axes in relation to the patient. The portability allows for the device to be moved around the operating theater or between theaters, and also for ease of storage. In embodiments having a rolling base 2150, once located in the desired position, the device can be secured to the bed or operating table. The support pole 2110 can have a mechanical means (resistance force mechanism 2610 is discussed further in reference to FIGS. 12A-12B; housing 2630 is visible in FIGS. 7B and 7D) to offset the weight of the tool, static arm 2130, swivel arm 2210, and tool holder 2260 such that raising and lowering of the scope requires near zero force, thereby substantially reducing the surgeon's fatigue. The entire procedure can be performed with the scope inserted in the device. Alternatively, the scope can be easily released from the device if needed.

A static arm 2130 is coupled horizontally to the top of the support pole 2110 at one end. The static arm 2130, also referred to as a boom, can extend over the patient. The static arm 2130 can be raised and lowered via the support pole. The raising and lowering capability allows for the user to accommodate for patients of various sizes and weights while still allowing for access to the operating field (e.g. between the area of the legs and abdomen in the case of a ureteroscopy). Static arm 2130 can have an arched architecture to traverse the patient such that the arm has clearance above the patient and drops the scope clamp down into the relevant surgical zone. In various embodiments, the static arm 2130 and support pole 2110 can be formed as a single part or can be coupled together with various coupling methods for joining poles such as fitted poles, interlocking poles, or couplers. Static arm 2130 and support pole 2110 can rotate as a unit within base 2150. In some embodiments, the static arm 2130 can communicate with the support pole 2110 via a vertical linear bearing sleeve to provide vertical height adjustment (not shown). The other end of the static arm 2130 is connected to a proximal end of swivel arm 2210. Swivel arm 2210 can rotate 360° about the y-axis at the connection to the static arm 2130. The swivel arm 2210 can be connected to the static arm 2130 by any suitable means allowing for rotation.

A tool holder 2260 can be coupled to a distal end of the swivel arm 2210. The tool holder 2260 is controlled by an actuator 2530. The tool holder can rotate along multiple axes of rotation and can be locked into any position when the actuator 2530 is engaged. In some embodiments, the rotatable joint 2510 is a ball-and-socket joint. In FIGS. 7A-8D, the tool holder 2260 is depicted holding a tool (e.g. a ureteroscope) 2270. The actuator 2530 can be controlled by an actuator controller 2550, shown here as a foot pedal. When a user steps on the foot pedal, the actuator 2530 causes the tool holder to lock, holding the tool in the desired position for a procedure. This allows for the surgeon to perform other tasks or access other tools without the need for repositioning the scope. In the depicted embodiment, the actuator controller 2550 is a foot pedal, but can be such as a button, trigger on the tool holder, a motion/gesture detector, or other controller means as can be envisioned by one of ordinary skill in the art. When the actuator controller 2550 is engaged a second time, the actuator 2530 is deactivated and the rotatable joint 2510 can again rotate.

In some embodiments, the swivel arm 2210 can have a pivot axis at both ends with the tool holder 2260 attached to the distal end, resulting in a linkage that allows the tool 2170 to be moved in free paths. The tool holder 2260 has six degrees of freedom such that it can move in x-, y-, and z-axis and rotate (yaw, roll, and pitch) around each of the 3 axes.

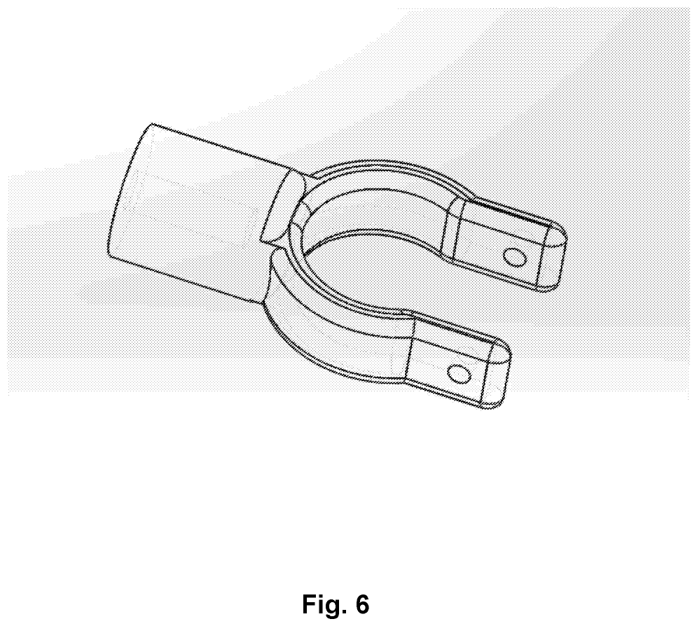

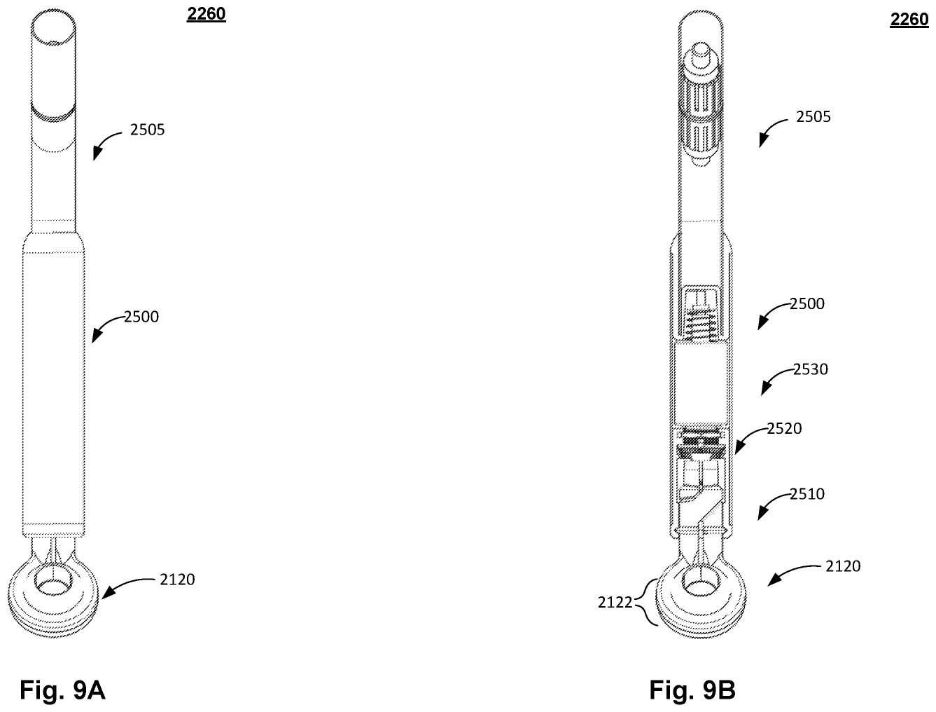

FIGS. 9A-9B, respectively, provide a front view and front cutaway view of one possible embodiment of a rotatable surgical tool holder 2260. The tool holder 2260 is shown without a tool. The tool holder has a rigid outer sleeve 2500 with a curved connecting portion 2505 at the proximal end and a clamp 2120 at the distal end. The clamp 2120 is attached to a rotatable joint 2510 having a locking mechanism 2520 in communication with an actuator 2530. The clamp 2120 is freely rotatable about the axis of the tool holder 2260 when the actuator 2530 is not actuated and is locked into position by a locking mechanism 2520 when the actuator 2530 is actuated. In the shown embodiment, the actuator 2530 is a solenoid. In the depicted embodiment, the clamp 2120 is formed by a pair of circular collars 2121. Other clamp 2120 shapes such as semi-circles or c-clamps (see FIG. 6) can be used as can be envisioned by one of ordinary skill in the art.

The curved connecting portion 2505 couples with the swivel arm 2210. In the shown embodiment, the curved connecting portion 2505 is a tube housing a shafted bearing assembly 2506 that allows the swivel arm 2210 to rotate. Other connectors allowing for rotational movement can be envisioned by one of ordinary skill in the art. In some embodiments, actuation of the actuator 2530 can also lock the swivel arm.

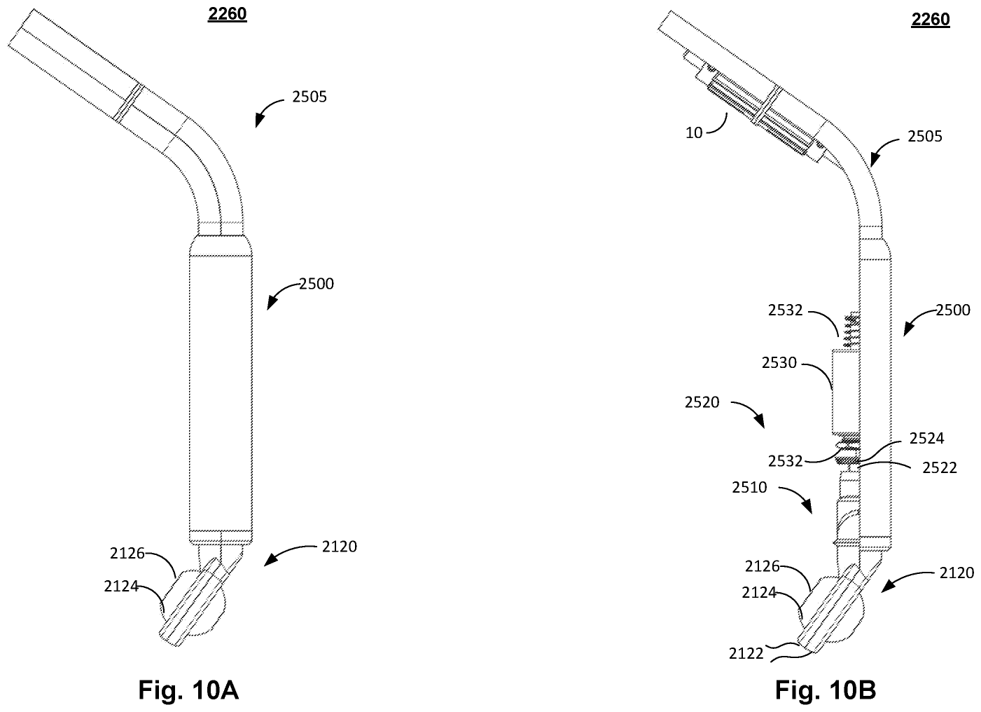

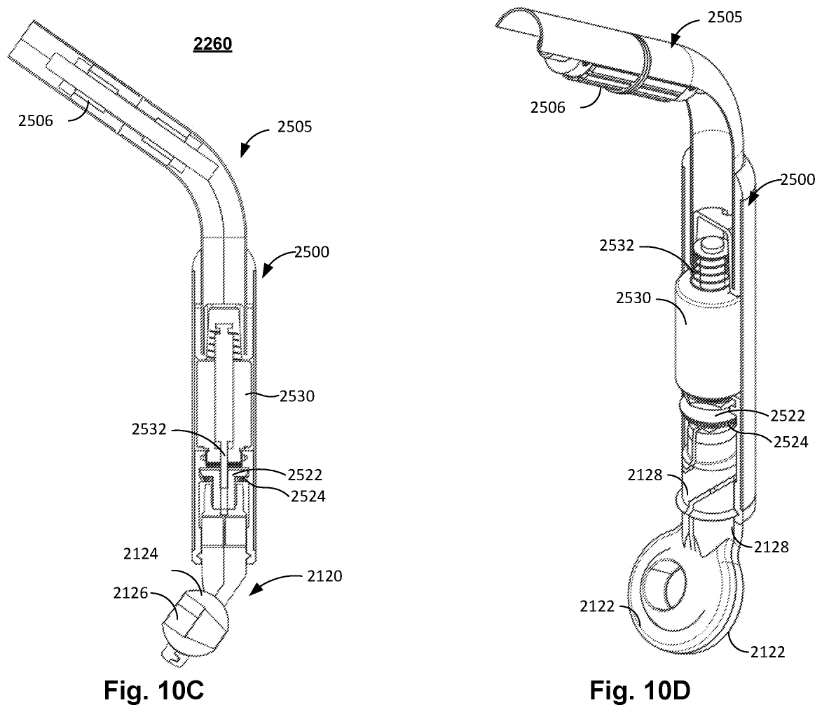

FIGS. 10A-10B, respectively, provide a side view and side cutaway view of the tool holder 2260 shown in FIGS. 9A-9B. In this embodiment, the clamp 2120 is shown with a tool seat 2124 between the clamp collars 2122. The tool seat 2124 is a sphere having an aperture 2126 for receiving a surgical tool. When the locking joint 2510 is not actuated, the sphere is rotatable in all degrees of freedom between the clamp collars 2122, such that a tool seated in the tool seat 2124 can be operated with joystick-like movement and control during positioning of the tool and for navigation within the patient's body. The tool seat aperture 2126 can be shaped to receive any tool. In some embodiments (not shown), a bespoke adapter for a specific tool can be fitted in a generic tool seat aperture 2126. In other words, the tool seat aperture 2126 can be custom fitted for a specific tool or the tool seat aperture 2126 can be a standard shape and size but can receive a custom tool seat adapter fitted for a specific tool.

An embodiment of the mechanism of the locking joint-clamp combination are shown in FIGS. 10C and 10D. The tool holder 2260 from a side cross-section view and a perspective cutaway view, respectively, is shown. FIG. 10C shows an embodiment with a spherical tool seat 2124 and FIG. 10D without the tool seat 2124 between clamp collars 2122. Clamp collars 2122 each end in a leg 2128. When the clamp collars 2122 are fitted together, the legs cross to form an X or scissor-like configuration to form a locking joint 2510. Each leg 2128 has a ridge which seats in a corresponding groove in outer sleeve 2500. When legs 2128 are spread, the ridge seats into the groove at the same time as clamp collars 2122 tighten. When the spherical tool seat 2124 is in place, this tightening of the clamp also works as a brake for any rotation of the tool seat 2124. The locking mechanism 2520 can be actuated by the actuator 2530. In the illustrated embodiment, actuator 2530 is a solenoid having a piston 2532 at each end. When actuated, the distal piston engages with the locking mechanism 2520 to lock the locking joint 2510. In the illustrated embodiment, locking mechanism 2520 includes a wedge 2522 having a circular proximal portion with radially oriented teeth 2524. When the locking mechanism 2520 is actuated, the solenoid piston presses on the wedge 2522, driving it in between the legs 2128 to expand the legs and tighten the clamp 2120. The teeth 2524 can be locked into place by engaging with a corresponding lip 2502 inside the outer sleeve 2500 to prevent rotation.

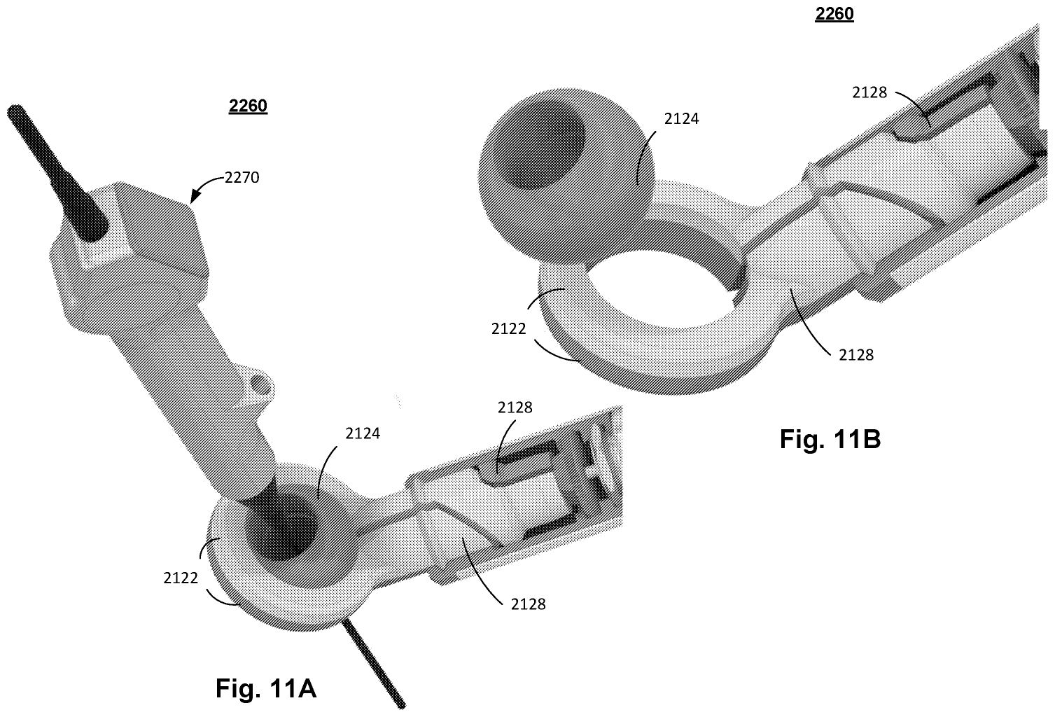

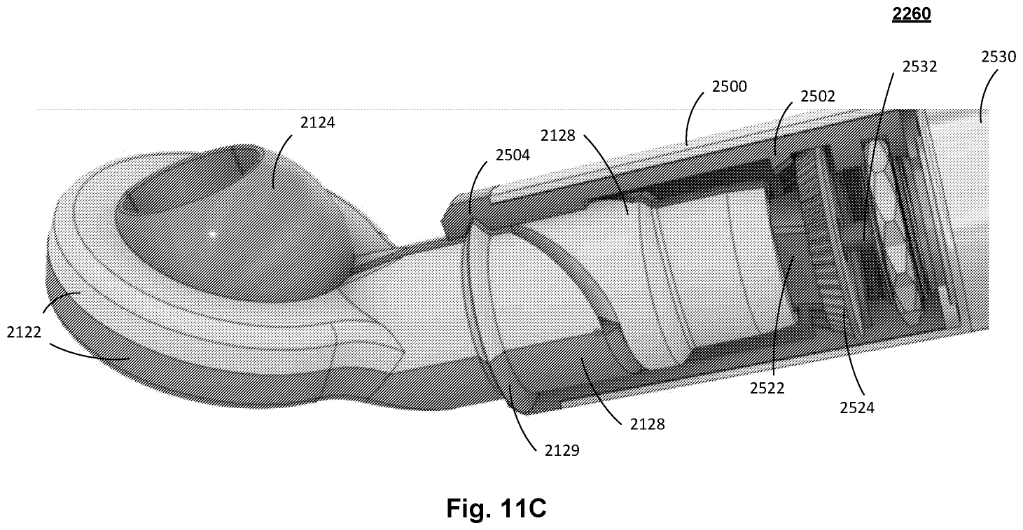

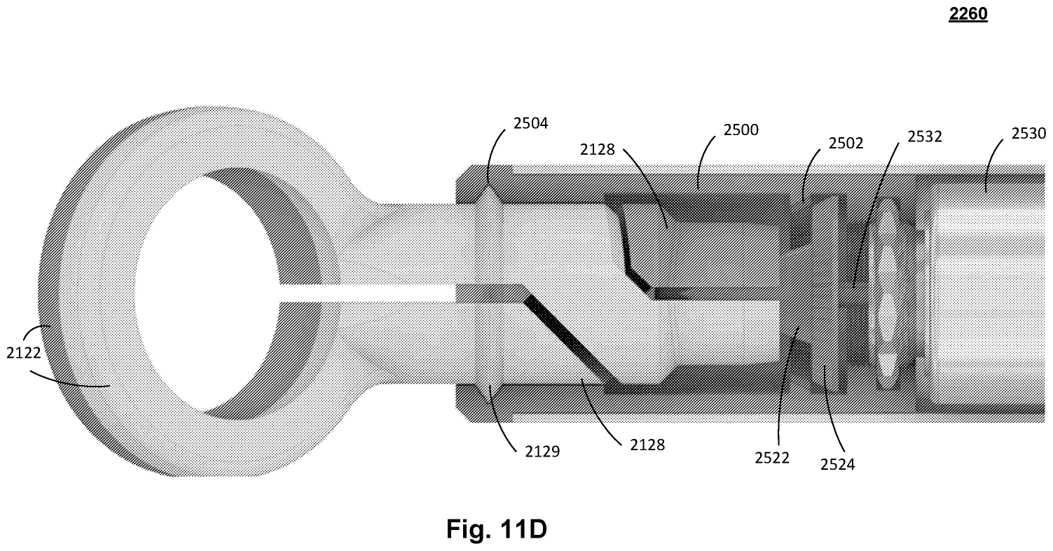

FIGS. 11A-11D provide shaded views of a surgical tool holder 2260 as described in FIGS. 10A-10D. The outer sleeve 2500 is shown cut away to illustrate the internal components. FIG. 11A shows tool 2270 seated in spherical tool seat 2124. FIG. 11Cs show the tool holder 2260 with spherical tool seat 2124 in place, and FIGS. 11B and 11D show the tool holder 2260 without tool seat 2124 to illustrate the locking joint 2510 and locking mechanism 2520. As can be seen, each clamp collar 2122 corresponds with a leg 2128. The corresponding collar-leg pairs are shown with one pair in light gray and one in dark gray. The legs 2128 can have a ridge 2129 that interacts with a corresponding groove 2504 (FIGS. 11C-11D).

FIGS. 12A-12C illustrate a possible counterbalancing mechanism of the surgical tool support system. The vertical support pole 2110 can be coupled to a resistance force mechanism 2610 to offset the combined weight of the tool and tool holder, static arm, and swivel arm such that the efforts needed for the user to raise and lower the tool requires near zero force, thereby substantially reducing the surgeon's fatigue. The resistance force mechanism 2610 can include a constant force coil spring 2620 having a resistance force to “uncoiling” that is equal to the weight and/or load of the components attached to the vertical support pole 2110. The constant force coil spring 2620 provides a constant offsetting of the force being required to raise or lower the scope. In some embodiments, the base 2150 into which the vertical support pole 2110 is inserted can include a housing 2630 to accommodate the resistance force mechanism 2610. FIG. 12C is a closeup of a model of the resistance force mechanism 2610 in which the constant force coil spring 2620 is wrapped beneath the support pole 2110 to provide a lift force equal to the load on the support pole 2110. Inclusion of the resistance force mechanism 2610 negates the force needed to move the scope, resulting in the surgeon needing only static activity to hold the scope rather than the dynamic activity of bearing the weight of the scope during movement.

Sterility in an operatory setting is important for preventing patient infection. All external parts of the devices and systems described herein can be wiped clean. In some embodiments, sleeves can be provided for the tool holder, the arms, or combinations thereof such the device can be wiped clean; sterile sleeves can be placed over the device prior to a procedure and removed afterward. In various embodiments, various parts can be disposable (e.g. the tool holder, tool seat, or clamp. Although not shown, the clamp and/or entire locking joint could be disposable and configured to be removably inserted, similar to a plug. In other embodiments, the tool holder can be autoclavable for reuse.

In some embodiments, the locking mechanisms described in Example 2 could be included at multiple joints of the device (e.g. swivel arm to static arm, swivel arm to tool holder, and/or in the tool holder) and controlled by the actuator such that the entire structure is locked with actuation. Alternatively, the ball and socket joints described in Example 1 may be used instead of or in conjunction with the joints in the device described in Example 2.

The surgical tool device and surgical tool holders described herein can be included in a surgical tool support system. The surgical tool support system can further include various options for providing easier access to tools and attachments during a surgical procedure. For example, the system can include an attachment for holding another tool (e.g. a laser) in a position relative to the scope. The attachment can be added to the tool holder or one of the arms. Additionally, other attachments such as wire holders or baskets, basket accessories, or other tools and or supplies can be coupled to the base, static arm, or support pole. The attaching can be permanent or removable/repositionable and can be such as hooks, clips, mesh baskets, or shelves.

### ASPECTS OF THE DISCLOSURE

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A surgical tool support device comprising a vertical support pole; a static arm coupled perpendicularly to the support pole at a first end; a swivel arm, wherein a proximal end of the swivel arm is rotationally coupled to a second end of the static arm; and a tool holder coupled to a distal end of the swivel arm, wherein the tool holder comprises a locking joint controlled by an actuator, wherein the tool holder can be locked into a position along multiple axes of rotation.

Aspect 2. The surgical tool support device of aspect 1, further comprising an actuator control, wherein the actuator control is selected from a foot pedal, button, trigger, or gesture control.

Aspect 3. The surgical tool support device of aspects 1 or 2, wherein the actuator is a solenoid or a motor brake.

Aspect 4. The surgical tool support device of any of aspects 1-3, wherein the support pole is configured to clamp to an operating table or bed.

Aspect 5. The surgical tool support device of any of aspects 1-3, wherein the support pole is affixed to a movable base.

Aspect 6. The surgical tool support device of any of aspects 1-5, wherein the tool holder comprises a surgical tool seat configured to receive a surgical tool, wherein the surgical tool seat is rotatable in multiple axes and wherein a position of the surgical tool seat is lockable via the actuator.

Aspect 7. The surgical tool support device of any of aspects 1-6, wherein a resistance force mechanism is coupled to the vertical support pole, wherein the resistance force mechanism provides an amount of force necessary to offset a weight of the tool holder and a tool such that near-zero force is required by a user to raise or lower the static arm.

Aspect 8. The surgical tool support device of any of aspects 1-7, wherein the resistance force mechanism comprises a constant force coil spring.

Aspect 9. The surgical tool support device of any of aspects 1-7, wherein the resistance force mechanism and the vertical support pole are housed in a base.

Aspect 10. A surgical tool holder comprising a clamp in communication with an actuator via a locking mechanism, wherein the clamp is freely rotatable around an axis of the tool holder when the acutator is not actuated and wherein the clamp is locked into position by a locking mechanism when the actuator is actuated.

Aspect 11. The surgical tool holder of aspect 10, wherein the actuator is a solenoid.

Aspect 12. The surgical tool holder of any of aspects 10 or 11, wherein a first end of the clamp comprises a collar and a second end of the clamp comprises scissored legs, and wherein the actuation causes the scissored legs to spread and the clamp to tighten.

Aspect 13. The surgical tool holder of any of aspects 10-12, wherein the clamp and locking mechanism are contained within a sleeve.

Aspect 14. The surgical tool holder of any of aspects 10-13, wherein the locking mechanism comprises a wedge having radial gear teeth, and wherein when actuated the wedge forces the scissored legs apart and wherein the gear teeth engage with a lip inside the sleeve such that the rotation of the clamp is locked.

Aspect 15. The surgical tool holder of any of aspects 10-14, further comprising a tool seat held by the clamp, wherein when the clamp is locked the tool seat is also locked.

Aspect 16. The surgical tool holder of aspect 15, wherein the tool seat is a sphere having an aperture configured to receive a surgical tool, and wherein when the clamp is unlocked, the sphere is rotatable in all degrees of freedom.

Aspect 17. A system for surgical tools comprising a surgical tool support device, the device comprising a vertical support pole, a static arm coupled perpendicularly to the support pole at a first end, a swivel arm, wherein a proximal end of the swivel arm is coupled to a second end of the static arm, and a tool holder coupled to a distal end of the swivel arm; the system further comprising at least one attachment, wherein the vertical support pole comprises resistance force mechanism to offset weight of the surgical tool for repositioning the surgical tool.

Aspect 18. The system of aspect 17, wherein the tool is a scope, further comprising an attachment for holding a second tool in a position relative to the scope.

Aspect 19. The system of aspects 17 or 18, further comprising at least one attachment for holding surgical tools or surgical supplies in reach of the user.

Aspect 20. The system of any of aspects 17-19, wherein the attachment is coupled to the surgical tool support device and is selected from a hook, a clip, a mesh basket, or a shelf.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that measurements, amounts, and other numerical data can be expressed herein in a range format. It is also understood that there are a number of values disclosed herein, and that each value is also herein disclosed as “approximately” that particular value in addition to the value itself. For example, if the value “10” is disclosed, then “approximately 10” is also disclosed. Similarly, when values are expressed as approximations, by use of the antecedent “approximately,” it will be understood that the particular value forms a further aspect. For example, if the value “approximately 10” is disclosed, then “10” is also disclosed.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

1. A surgical tool support device comprising:
a vertical support pole;
a static arm coupled perpendicularly to the support pole at a first end;
a swivel arm, wherein a proximal end of the swivel arm is rotationally coupled to a second end of the static arm; and
a tool holder coupled to a distal end of the swivel arm, wherein the tool holder comprises a locking joint controlled by an actuator, wherein the tool holder can be locked into a position along multiple axes of rotation.

2. The surgical tool support device of claim 1, further comprising an actuator control, wherein the actuator control is selected from a foot pedal, button, trigger, or gesture control.

3. The surgical tool support device of claim 1, wherein the actuator is a solenoid or a motor brake.

4. The surgical tool support device of claim 1, wherein the support pole is configured to clamp to an operating table or bed.

5. The surgical tool support device of claim 1, wherein the support pole is affixed to a movable base.

6. The surgical tool support device of claim 1, wherein the tool holder comprises a surgical tool seat configured to receive a surgical tool, wherein the surgical tool seat is rotatable along multiple axes and wherein a position of the surgical tool seat is lockable via the actuator.

7. The surgical tool support device of claim 1, wherein a resistance force mechanism is coupled to the vertical support pole, wherein the resistance force mechanism provides an amount of force necessary to offset a weight of the tool holder and a tool such that near-zero force is required by a user to raise or lower the static arm.

8. The surgical tool support device of claim 7, wherein the resistance force mechanism comprises a constant force coil spring.

9. The surgical tool support device of claim 7, wherein the resistance force mechanism and the vertical support pole are housed in a base.

10. A surgical tool holder comprising:
a clamp in communication with an actuator and
a locking mechanism, wherein:
the clamp is freely rotatable around an axis of the tool holder when the acutator is not actuated; and
the clamp is locked into position by the locking mechanism when the actuator is actuated.

11. The surgical tool holder of claim 10, wherein the actuator comprises a solenoid.

12. The surgical tool holder of claim 10, wherein a first end of the clamp comprises a collar and a second end of the clamp comprises scissored legs, and wherein the actuation causes the scissored legs to spread and the clamp to tighten.

13. The surgical tool holder of claim 12, wherein the clamp and locking mechanism are contained within a sleeve.

14. The surgical tool holder of claim 13, wherein the locking mechanism comprises a wedge having radial gear teeth, and wherein when actuated the wedge forces the scissored legs apart and wherein the gear teeth engage with a lip inside the sleeve such that the rotation of the clamp is locked.

15. The surgical tool holder of claim 12, further comprising a tool seat held by the clamp, wherein when the clamp is locked the tool seat is also locked.

16. The surgical tool holder of claim 15, wherein the tool seat is a sphere having an aperture configured to receive a surgical tool, and wherein when the clamp is unlocked, the sphere is rotatable in all degrees of freedom.

17. A system for surgical tools comprising:
a surgical tool support device comprising;
a vertical support pole;
a static arm coupled perpendicularly to the support pole at a first end;
a swivel arm, wherein a proximal end of the swivel arm is coupled to a second end of the static arm; and
a tool holder coupled to a distal end of the swivel arm; and
at least one attachment;
wherein the vertical support pole comprises resistance force mechanism to offset weight of the surgical tool for repositioning the surgical tool.

18. The system of claim 17, wherein the tool is a scope and wherein the attachment holds a second tool in a position relative to the scope.

19. The system of claim 17, wherein the at least one attachment holds surgical tools or surgical supplies in reach of the user.

20. The system of claim 19, wherein the attachment is coupled to the surgical tool support device and is selected from a hook, a clip, a mesh basket, or a shelf.
